(19) United States
(12) Patent Application Publication
(10) Pub. No.: US 2008/0262437 A1
(43) Pub. Date: Oct. 23, 2008

(54) DOSE SETTING MECHANISM WITH OVERSTRAIN LOCK

(76) Inventors: Juerg Hirschel, Aarau (CH); Ulrich Moser, Heimiswil (CH); Christian Schrul, Burgdorf (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/052,906

(22) Filed: Mar. 21, 2008

(30) Foreign Application Priority Data
Mar. 22, 2007 (DE) 102007014426.3
Oct. 24, 2007 (DE) 202007014852.6

(51) Int. Cl. A61M 5/31

(52) U.S. Cl. 604/207

(57) ABSTRACT

A setting mechanism for setting or selecting a quantity of a substance to be dispensed from an injection device, the mechanism including a dose setting element and an adjusting element which can be operated by a user, wherein the adjusting element is operably coupled to the dose setting element and an overstrain lock is provided between the dose setting element and the adjusting element.

### CROSS-REFERENCE TO RELATED APPLICATIONS

This application claims priority to German Application No. DE 20 2007 014 852.6 filed on Oct. 24, 2007 and German Application No. DE 10 2007 014 426.3 filed on Mar. 22, 2007, the contents of both of which are incorporated in their entirety herein by reference.

### BACKGROUND

The present invention relates to devices for injecting, infusing, administering, delivering or dispensing a substance, and to methods of making and using such devices. More particularly, it relates to a setting mechanism for preparing or setting a quantity or dose of a substance to be administered from an injection device, such as insulin for example.

Injection devices for administering set or selected doses of a substance from a supply chamber or from an ampoule inserted in the injection device are known from the prior art. To set the exact quantity or dose of the substance contained in the injection device representing the quantity of substance that will be dispensed during an injection, the quantity of substance to be dispensed may be set by an adjusting element, which can be operated by a user and is usually a rotatable adjustable element. A set amount usually depends on the degree of the rotating movement, in other words the rotation angle of the adjusting element provided on the injection device. This being the case, the adjusting element can be rotated about the longitudinal axis of the injection device and, depending on the design of the injection device, the adjusting element is pushed or moved out of the injection device in the axial or longitudinal direction and screwed out for example, or alternatively is not pushed in the axial direction.

A stop is usually provided to indicate a maximum dose or set a maximum dose, whereby a cam of an element connected to the adjusting element projecting radially outwardly is rotated onto a stop projecting radially inwardly which is not able to move during the setting movement and restricts the maximum dose. Turning the adjusting element any further would cause the mutually abutting stops or lugs to break or be deformed in the fully fitted state, for example.

### SUMMARY

An object of the present invention is to provide an improved setting mechanism for setting or selecting a quantity of a substance to be dispensed from an injection device by which a maximum possible setting quantity can be predefined.

In one embodiment, the present invention comprises a setting mechanism for setting or selecting a quantity of a substance to be dispensed from an injection device, the mechanism comprising a dose setting element and an adjusting element which can be operated by a user, wherein the adjusting element is operably coupled to the dose setting element, the mechanism further comprising an overstrain lock between the dose setting element and the adjusting element.

In some preferred embodiments, in accordance with the present invention an adjusting mechanism for adjusting, selecting or setting a quantity or dose of substance to be dispensed from an injection device has a dose setting element which is able to slide and/or rotate relative to the injection device and which can be moved and/or rotated relative to the injection device, for example about the longitudinal axis of the injection device. The dose setting element is coupled with the injection device so that the quantity of the substance to be dispensed from the injection device is set depending on the angle of rotation, for example the number of rotations, of a dose setting sleeve serving as a dose setting element, which is rotated starting from a defined zero or initial state during the dose setting operation. This being the case, as the quantity of substance to be dispensed is higher or larger, the more or the further the dose setting element is rotated.

In most known injection devices with a setting feature, there is a proportional relationship between the rotation of the dose setting element and the quantity of substance to be dispensed. The dose setting element may be designed so that it is pushed in the axial direction of the injection device during an adjusting movement or rotation and is turned out or screwed out, for example, and the axial offset of the dose setting element relative to the injection device during a dose setting or adjusting operation determines the plunger stroke or distance traveled by a displaceable stopper in an ampoule, for example, thereby determining the quantity of substance to be dispensed. The dose setting element is coupled with an adjusting element, which can be operated by a user in such a way that an adjusting movement or rotation of the adjusting element within a pre-definable dose setting range, starting from a zero position to a rotation position corresponding to less than a maximum dose, is transmitted to the dose setting element, for example.

In accordance with the present invention, an overstrain lock is provided between the dose setting element and the adjusting element, which prevents or reduces transmission of a force or the transmission of a movement from an adjusting element operated by the user to the dose setting element if the dose setting element should not be rotated any further, for example because a maximum dose has been set. In some embodiments, a cam of the dose setting element lies against or abuts a stop which remains stationary in the injection device during the setting operation and is provided for restricting the dose which can be set, for example, and prevents any further rotation of the dose setting element.

In some exemplary embodiments, the dose setting element may be provided in the form of a dose setting sleeve which is cylindrical in shape or has a sleeve-shaped or cylindrical region and extends into the injection device. The adjusting element may be provided in the form of a dose setting ring, which has a ring-shaped or disc-shaped element, connected to a sleeve-shaped projection which may be pushed into a sleeve-shaped region of the dose setting element.

In one embodiment, the adjusting element, in other words the dose setting ring, can be rotated relative to the dose setting element when a relative force or a torque between the adjusting element and the dose setting element exceeds a minimum value predefined by the structural design. A rotating movement of the adjusting element can be transmitted to the dose setting element if the relative force or torque between the dose setting element and adjusting element is below the maximum force or maximum torque predefined by the structural design, which is preferably lower than the retaining force or a maximum torque still permitting transmission by which the dose setting element is retained in the injection device in a position turned out to the maximum so that it is prevented from turning further. When the adjusting element is rotated by an angle of 390° for example, this rotating movement of the adjusting element is transmitted to the dose setting element so that the dose setting element is also rotated by 390°. It is not until a predefined restriction occurs due to the structural design that the dose setting element is retained by this restriction and further rotation of the dose setting element is prevented, as a result of which the coupling between the dose setting element and the adjusting element is released if the adjusting element is rotated any further and a rotation of the adjusting element can no longer be transmitted to the dose setting element, and the dose setting element and the adjusting element are coupled with one another by a positive fit so that they do not release from one another, even though a rotating movement can no longer be transmitted. In the event of a subsequent adjusting movement or an attempt to increase the set dose, therefore, the adjusting mechanism or the injection device can not be damaged.

In some embodiments, the coupling between the dose setting element and the adjusting element may be achieved on the basis of an elastic material in the region of the coupling to establish a positive connection between the dose setting element and adjusting element. The positive connection still permits a relative movement between the dose setting element and the adjusting element which lies in a direction that does not correspond to the setting direction. If the setting direction is a direction of rotation, in other words is in a circumferential direction or motion, the coupling of the dose setting element with the adjusting element may be designed so that the adjusting element can be pushed in a direction that is different from the setting direction, in other words in an axial direction of the dose setting element or adjusting element, along an axis constituting a mid-axis of the rotation or setting movement. To this end, a surface may be provided on the dose setting element and/or adjusting element which extends at an angle by reference to the setting direction or axial direction of the dose setting element or adjusting element. The surface may be designed as a conically tapering element or annular cut-out of a cone surface. If an elastic material is used in the region of the coupling or contact surface between the dose setting element and the adjusting element, this will permit a relative movement due to a deformation of the elastic material which, in the absence of the external force which led to this relative movement, will result in an automatic backward or reverse movement of the adjusting element on or in the dose setting element.

In one embodiment, an annular snapper bead or raised area may be provided on one of the elements which engages in a snapper groove of the other element. For example, a snapper bead may be on a cylindrically shaped projection of or associated with the dose setting element, which locates in a snapper groove extending circumferentially around the internal face of the dose setting element. This being the case, the snapper bead and/or the snapper groove may have a conical region.

In some preferred embodiments, at least one and possibly several locking catch and/or co-operating catch elements may be provided in the circumferentially extending direction on the contact surface or contact region between the dose setting element and the adjusting element. The contact region between the dose setting element and the adjusting element on which these elements may be disposed may be a top region or end region of the dose setting element which comes into contact with a button or the disc-shaped region of the adjusting element. The locking or catch elements and co-operating elements have a mutually corresponding geometry. For example, triangular projecting catch cams may be provided on one element, which are able to engage in corresponding triangular recesses on the other element and which can be disengaged by a movement of the adjusting element out of the dose setting element while the dose setting element is being held and the adjusting element continues to be operated or rotated to enable the adjusting element to rotate relative to the dose setting element.

### BRIEF DESCRIPTION OF THE DRAWINGS

FIG. 1A is a plan view of an embodiment of an injection device in accordance with the present invention in one operational position;

FIG. 1B is a sectional view taken along line A-A of FIG. 1A;

FIG. 2A shows the injection device illustrated in FIG. 1A in a charged position with the adjusting and dose setting elements rotated outwardly;

FIG. 2B is a sectional view along line B-B of FIG. 2A;

FIG. 3 is a sectional view along line C-C of FIG. 2A;

FIGS. 4A, B and C show a perspective view, side view and a sectional view of the dose setting element;

FIGS. 5A, B and C show a perspective view, a plan view and a side view of the adjusting element;

FIG. 6 is a perspective view of a portion of one embodiment of an injection device in accordance with the present invention; and

FIG. 7 is an exploded perspective, including details drawn thereform, showing an embodiment of a dose selection ring with four snapper arms able to move inwardly and outwardly, each of which has catch elements pointing radially inwardly and outwardly.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making the invention and/or its components may be selected from appropriate materials such as metal, metallic alloys, ceramics, plastics, etc.

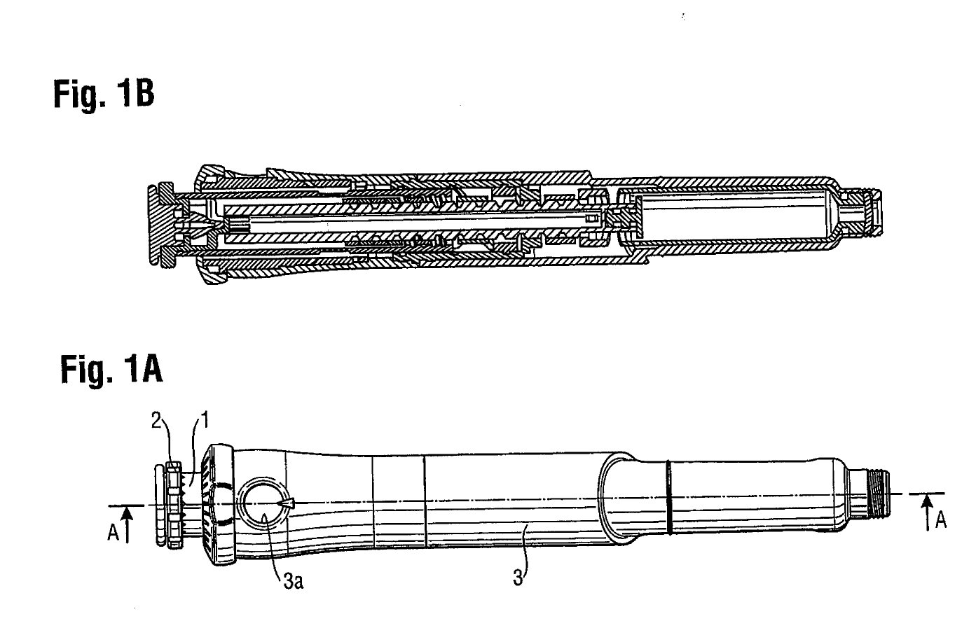

FIG. 1B shows a view of the injection device 3 in cross-section along line A-A indicated in FIG. 1A, in which the dose setting sleeve 1 serving as the dose setting element, which is coupled with the dose setting ring 2 fitted on it or in it so that it can not rotate, is pushed in. The injection device 3 is therefore in an initial position.

For details of the structure and the way in which a dose setting mechanism and an injection device or pen with such a dose setting mechanism inserted in it operates, reference may be made to patent specification DE 10 2005 044 096 A1, which is included in the teaching of the present application by way of reference.

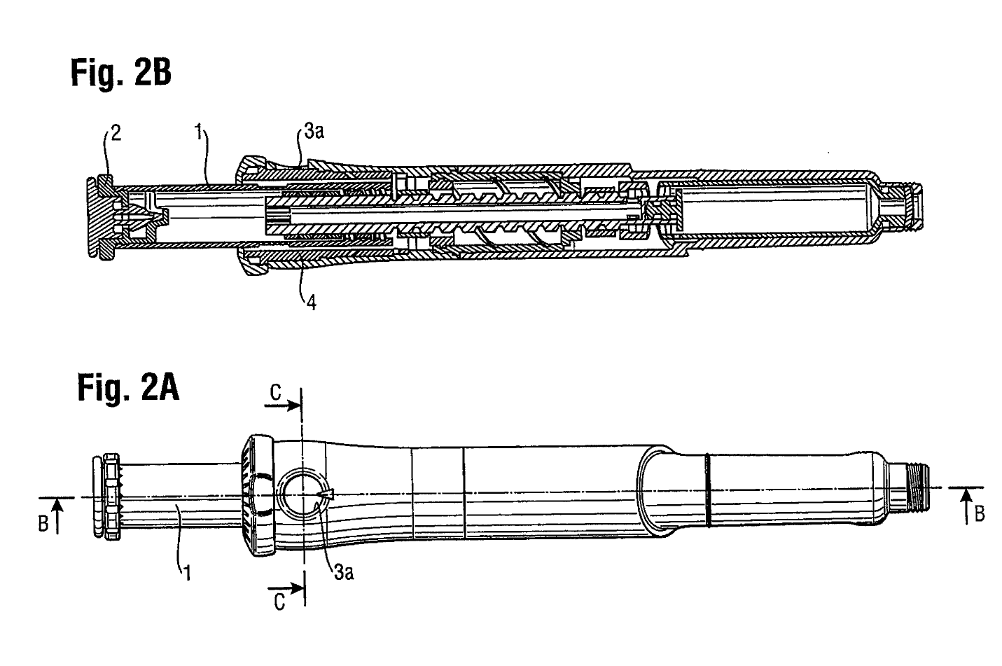

If the dose setting sleeve 1, which has a thread on its external face engaging in an internal thread 3d of the injection device 3 or a part (guiding sleeve) fixedly joined to the housing, is screwed out by a rotating movement of the dose setting ring 2 coupled with the dose setting sleeve 1, the injection device 3 is in a charged position as illustrated in FIG. 2A.

As may be seen from the view in cross-section illustrated in FIG. 2B, the dose selection ring 4 has a display on its external face with numbers applied to it, so that the size of a set dose can be read through a viewing window 3a provided in the housing of the injection device 3.

The dose selection ring 4 can be rotated relative to the injection device 3 or housing of the injection device 3 so that the stop 4a of the dose setting ring 4 pointing radially inwardly is turned as a function of the rotating movement of the dose selection ring 4 to fix the maximum dose based on the position of rotation of the stop 4a. The adjusted position of rotation of the dose selection ring 4 may be locked by a lock mechanism when the dose setting sleeve 1 is pulled out of the injection device by a small amount.

Provided on the internal circumference of the housing extending in the axial direction are grooves along which the catch elements mounted on snapper or resilient arms of the dose selection ring 4 and pointing radially outwardly are able to move, thereby generating a clicking noise.

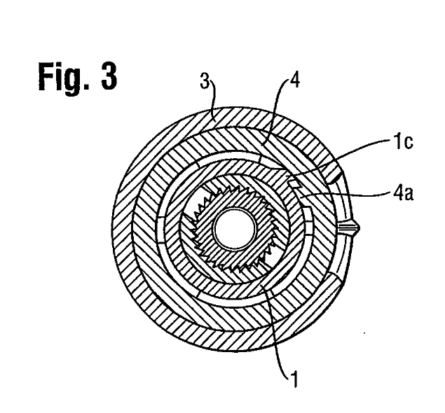

As may be seen from FIG. 3, the dose setting sleeve 1 is mounted or positioned inside the dose selection ring 4. During the adjusting operation, the dose setting sleeve 1 is moved backward or rearwardly in the axial direction and moved or rotated out of the injection device.

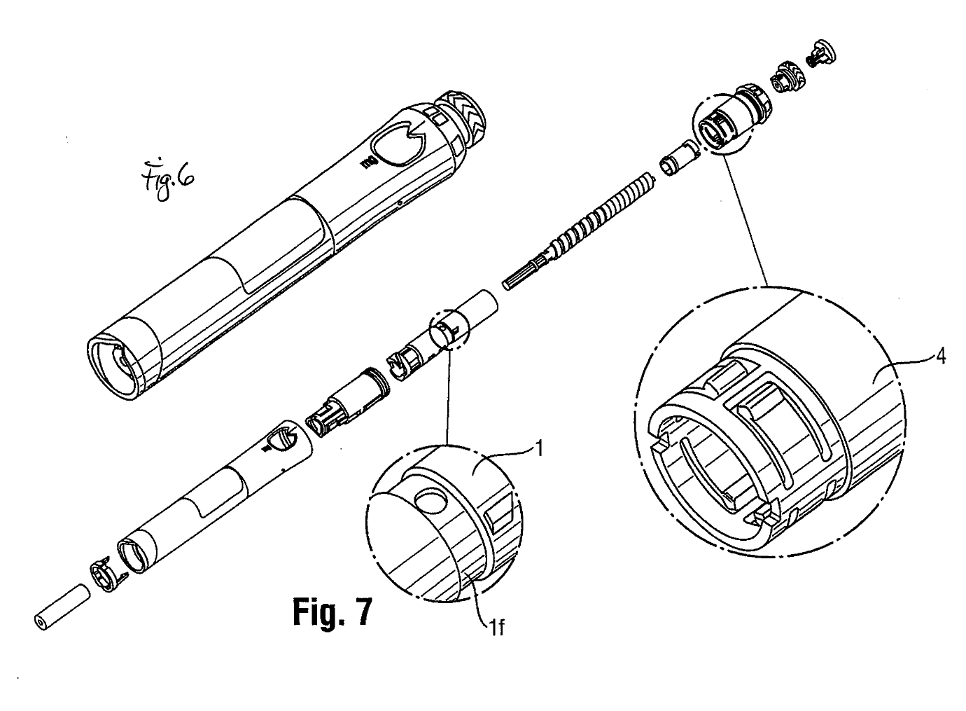

FIG. 6 is a perspective view of an embodiment of an injection device in accordance with the present invention, and FIG. 7 is an exploded perspective view, including a view of a middle and/or front region of the dose setting sleeve 1. A circumferentially extending groove 1f is provided in which the catch elements of the dose selection ring 4 pointing radially inwardly lie in the position illustrated in FIG. 1. The resilient arms of the dose selection ring 4 are therefore able to flex radially inwardly at a transition from one axially extending groove of the housing to the next one, which is what enables the rotating movement of the dose selection ring 4 while simultaneously generating clicking noises.

When, having primed a dose set by the dose selection ring 4, the dose setting sleeve 1 is pulled out of the injection device and hence out of the dose selection ring, the radially extending groove 1f of the dose setting sleeve 1 no longer lies in the region of the snapper arms of the dose selection ring 4 and the catch elements pointing radially outwardly are therefore no longer able to flex inwardly and couple the dose selection ring with the injection device to prevent it from rotating due to an engagement in the radially extending inner grooves of the injection device.

By the dose selection ring 4, the dose to be dispensed can be set inside the injection device 3 and when the dose setting sleeve 1 is fitted, a cam or a lug 1c of the dose setting sleeve 1 projecting radially outwardly is turned onto the stop 4a of the dose selection ring 4 projecting radially inwardly, as may be seen from FIG. 3. If the dose setting sleeve 1 were rotated further, the mutually abutting stops or lugs 1c and 4a of the dose setting sleeve 1 and the dose selection ring 4 respectively could break or be deformed in the fully attached state.

However, since the user does not operate the dose setting sleeve 1 directly and instead operates the dose setting ring 2 coupled with the dose setting sleeve 1, which can be snap-fitted onto the dose setting sleeve 1 for example, the stops 1c and 4a are prevented from breaking or deforming.

During a setting operation, the dose setting ring 2 is operated by a user, for example held. The ring has a snapper bead 2a extending around its circumference which snaps into a snapper groove 1a on the internal face of the dose setting sleeve 1. Projecting in the axial direction of the injection device or pen 3, triangular catch cams 2c are provided extending circumferentially around the dose setting ring 2, which latch in corresponding catch grooves 1d in the proximal external circumferential face of the dose setting sleeve 1. When the dose setting ring 2 is rotated further, even though the dose setting sleeve 1 lies with its cam 1c against the inner cam 4a of the dose selection ring 4, the catch cams 2c of the dose setting ring 2 are released from the catch grooves 1d of the dose setting sleeve 1 and rotated by one position. As this happens, the ramp or a surface on the snapper bead 2a of the dose setting sleeve 2 causes a biasing movement of the dose setting ring 2 into the dose setting sleeve 1 so that, following a further movement of the triangular catch cams 2c of the dose setting ring 2, the catch cams 2c are able to latch in the catch grooves 1d of the dose setting sleeve 1 again in the next position.

This results in an overstrain lock, i.e. the dose setting sleeve 1 can no longer be over-rotated relative to the dose selection ring 2 and the stops 1c and 4a can no longer be deformed or broken.

It is of advantage if the biasing action of the dose setting ring 2 is set so that the triangular catch cams 2c of the dose setting ring 2 can be more easily released from the catch grooves 1d of the dose setting sleeve 1, i.e. with a lighter force, than a force which would be necessary to deform or break the stops 1c and 4a of the dose setting sleeve 1 and the dose selection ring 4.

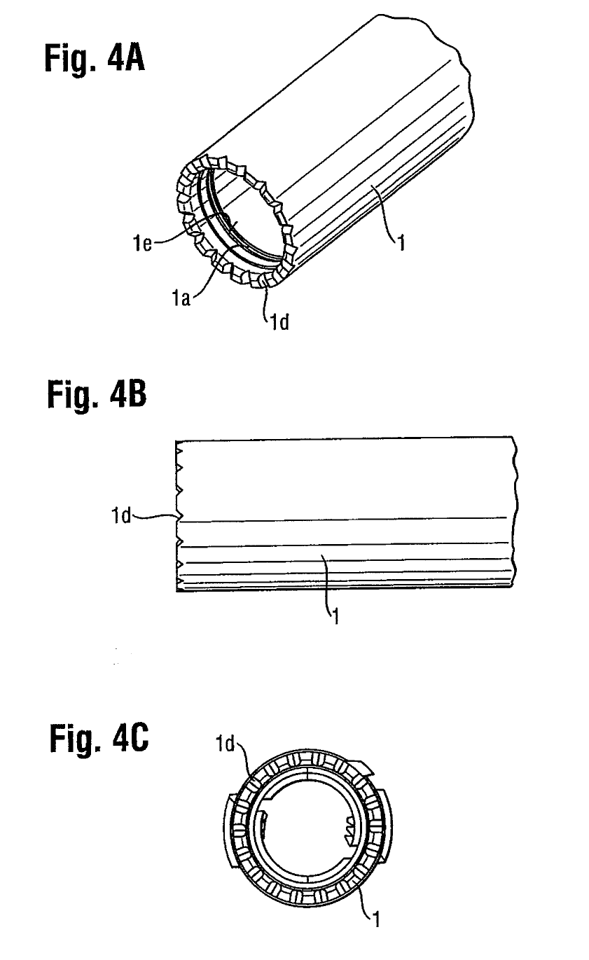

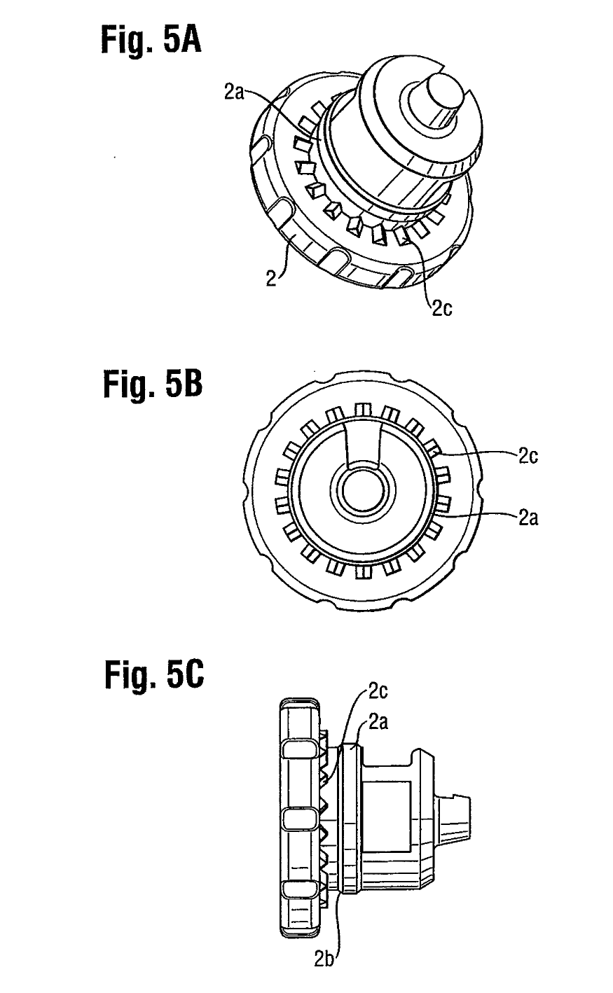

FIG. 4a shows a perspective view of the dose setting sleeve 1 with the snapper groove 1a in the internal face extending in the circumferential direction, in which the snapper bead 2a of the dose setting ring 2 engages. Extending around the circumference of its axial boundary surface, the dose setting sleeve 1 has several triangular recesses 1d pointing in the axial direction, in which the catch cams 2c of the dose setting ring 2 illustrated in FIG. 5A are able to engage.

As may be seen from FIG. 5C, the snapper bead 2a has an oblique or conical region 2c, which is able to lie against the conical region 1e of the snapper groove 1a illustrated in FIG. 4A. The snapper bead 2a and/or the dose setting sleeve 1 in the region of the snapper groove 1a are made from an elastic material to permit an outward movement of the dose setting ring 2. The movement is sufficient so that the anti-rotation coupling established by the catch cams 2c and the catch grooves 1d can be at least temporarily released.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A setting mechanism for setting or selecting a quantity of a substance to be dispensed from an injection device, the mechanism comprising a dose setting element and an adjusting element which can be operated by a user, wherein the adjusting element is operably coupled to the dose setting element, the mechanism further comprising an overstrain lock between the dose setting element and the adjusting element.

2. The setting mechanism as claimed in claim 1, wherein the dose setting element and the adjusting element each comprise at least one catch element, said catch elements co-operating to releaseably, non-rotatably couple the dose setting and adjusting elements.

3. The setting mechanism as claimed in claim 2, wherein the catch elements are circumferentially distributed around a circumference of the dose setting element and the adjusting element whereby the non-rotational coupling can be re-established after a rotation of the adjusting element relative to the dose setting element by less than 360°.

4. A setting mechanism for providing a quantity of a substance to be dispensed from an injection device, the setting mechanism comprising a dose setting element and an adjusting element which can be operated by a user and which is coupled with the dose setting element, wherein an overstrain lock is provided between the dose setting element and the adjusting element.

5. The setting mechanism as claimed in claim 4, wherein the dose setting element and/or the adjusting element are one of generally cylindrical or annular.

6. The setting mechanism as claimed in claim 4, wherein the adjusting element is prevented from rotating relative to the dose setting element and can be rotated when a force is applied.

7. The setting mechanism as claimed in claim 4, wherein the coupling between the dose setting element and the adjusting element is such that the adjusting element can be moved in an axial direction relative to the dose setting element against a rebound force.

8. The setting mechanism as claimed in claim 7, wherein the movement in the axial direction is restricted in length.

9. The setting mechanism as claimed in claim 4, wherein at least one of the dose setting element and the adjusting element comprise an elastic material in the region of the coupling.

10. The setting mechanism as claimed in claim 4, wherein the dose setting element and the adjusting element are positively coupled with one another.

11. The setting mechanism as claimed in claim 4, wherein at least one of the dose setting element and the adjusting element have a conical or frustoconical region to establish a positive fit.

12. The setting mechanism as claimed in claim 4, wherein the dose setting element and the adjusting element each have at least one releasable co-operating catch element, said catch elements co-operating to releaseably couple the adjusting element and the dose setting element against rotating.

13. The setting mechanism as claimed in claim 12, wherein the catch elements are distributed around a circumferential or annularly shaped portion of the dose setting element and adjusting element so that an anti-rotation coupling can be re-established after a rotation of the adjusting element relative to the dose setting element by less than 360°.
